(19) United States
(12) Patent Application Publication
(10) Pub. No.: US 2024/0194343 A1
(43) Pub. Date: Jun. 13, 2024

(54) PAIN DETECTION VIA MACHINE LEARNING APPLICATIONS

(71) Applicant: HERO Medical Technologies Inc., Ponte Vedra Beach, FL (US)

(72) Inventors: Deborah Eve Kantor, Ponte Vedra Beach, FL (US); Elliot Kantor, Ponte Vedra Beach, FL (US)

(73) Assignee: HERO Medical Technologies Inc., Ponte Vedra Beach, FL (US)

(21) Appl. No.: 18/531,800

(22) Filed: Dec. 7, 2023

Related U.S. Application Data
(60) Provisional application No. 63/431,595, filed on Dec. 9, 2022.

(51) Int. Cl. G16H 50/20; G06N 3/08; G16H 50/50

(52) U.S. Cl. CPC G16H 50/20; G06N 3/08; G16H 50/50

(57) ABSTRACT

The technical solutions identify presence and the level of pain of a patient using ML modeling trained on media data and data on health of persons. A processor coupled with memory can receive, from an application, a media data and data on health of the person. The processor can identify, using ML models, a presence of pain and a level of pain being expressed in the media of the person responsive to providing the media and the data on health of the person as inputs to the ML models. The ML models can be trained using a plurality of media and a plurality of data on health of persons expressing a plurality of levels of pain. The processor can generate, for the application, a notification identifying the presence of pain and the level of pain expressed by the person.

### CROSS-REFERENCE TO RELATED PATENT APPLICATIONS

This application claims the benefit of and priority to U.S. Provisional Application No. 63/431,595, filed Dec. 9, 2022, which is incorporated herein by reference in its entirety and for all purposes.

### FIELD OF THE DISCLOSURE

This disclosure generally relates to detection of pain using machine learning, including without limitation, systems and methods for detection of pain using machine learning models trained on media and medical information datasets.

### BACKGROUND

Patients suffering injury or trauma can experience different kinds and degrees of pain, which can be assessed in accordance with different ratings and scales. Pain assessment can be an important factor in improving the treatment of patients. However, timely assessment of different types of pain for different patients remains a challenge.

### SUMMARY

The technical solutions of this disclosure are directed to systems and methods that utilize machine learning (ML) or artificial intelligence (AI) modelling and user device applications to detect, assess or quantify pain experienced by individual patients. For instance, the technical solutions can facilitate detection, assessment, quantification or characterization of pain experienced by any patient, such as injured military service members with battlefield and training related injuries, patients injured in vehicular accidents, patients suffering from diseases, such as rheumatoid arthritis, or any other painful condition. Various injuries can hinder physical capabilities of individuals to different degrees. In many instances, experienced pain remains underreported. For example, battlefield and training-related injuries can challenge force readiness of injured military personnel by causing pain that can interfere with various military personnel duties. Among service members with mild Traumatic Brain Injury (mTBI), about 48% of injuries remain unreported even though about 91% of such injuries cause pain interference. Moreover, different persons can experience and tolerate pain differently, it is challenging to determine an accurate level of pain based on objective data.

The technical solutions of this disclosure overcome these challenges by providing ML or AI models trained using large datasets that can include media data (e.g., videos or images), sensor data, or documents (e.g., files or documented data) corresponding to the health of a patient, to provide a quick and efficient pain assessment and diagnosis. In doing so, the technical solutions can facilitate more accurate detection and reporting of under-reported pain conditions, such as, for example, pain associated with head injuries. The ML or AI models of the technical solution can be trained using prior, retrospective or prospective data. The data can include images and/or videos of persons, such as images or videos of faces, hands, arms, fingers, torso, legs, or various motions or movements of persons experiencing pain. The prior, retrospective or prospective data can include, for example, any number of publicly available images of persons experiencing pain, patients with head injuries and health related data, such as vital sign data of the patients. The data can include anywhere from several tens to several hundreds of thousands of pieces of data or images. The present solution can utilize the bench-test image capture and vital signal inputs as data to train or input into the ML models to predict or assess pain. A python-based mobile application interacting with one or more neural-network trained ML models can be provided to a user to determine the pain level being experienced by one or more persons. The pain determinations can be made using or according to the image data of a patient as well as the patient's data, such as for example the patient's age, medical history and the current vital sign data.

At least one aspect is directed to a system. The system can include one or more processors coupled with memory to receive, from an application, a media of a person and data on health of the person. The one or more processors can be configured to identify, using one or more models, a presence of pain and a level of pain being expressed in the media of the person responsive to providing the media of the person and the data on health of the person as one or more inputs to the one or more models. The one or more models can be trained using a plurality of media and a plurality of data on health of persons expressing a plurality of levels of pain. The one or more processors can be configured to generate, for the application, a notification identifying the presence of pain and the level of pain expressed by the person.

The one or more processors can be configured to train the one or more models to identify the presence of pain using at least the plurality of media. The plurality of media can comprise at least one of a plurality of videos or a plurality of images of one or more body parts of a plurality of body parts of the plurality of persons expressing pain and not expressing pain. The one or more processors can be configured to train the one or more models to identify the level of pain based at least on the plurality of data. The plurality of data can comprise vital sign data of the plurality of persons experiencing the plurality of levels of pain.

The one or more processors can be configured to determine the level of pain of a plurality of levels of pain of the person based at least on the data on health including least one biometric data point such as that of a heart rate, temperature, oxygen level (SpO₂), respiratory rate, a systolic blood pressure, and age of the person input into the one or more models. The one or more processors can be configured to determine, using the data on health input into a model of the one or more models comprising a neural network, at least one of the presence of pain or the level of pain. The data on health can comprise at least one of a medical history of the person or a measurement of a sensor attached to a body of the person.

The one or more processors can be configured to identify, from the received media, at least one of a video or an image of the received media depicting a portion of a face of the person. The one or more processors can be configured to identify, using the one or more models, at least one of the presence of pain or the level of pain responsive to providing the at least one of the video or the image as an input of the one or more inputs to the one or more models.

The one or more processors can be configured to receive the media comprising a video capturing a movement of a plurality of parts of a body of the person. The one or more processors can be configured to identify, using the one or more models, at least one of the presence of pain or the level of pain responsive to the movement. The one or more processors can be configured to receive the data on health comprising at least one of a prospective measurement or a retrospective measurement of a sensor of a wearable device of the person. The one or more processors can be configured to identify at least one of the presence of pain or the level of pain responsive to providing the at least one of the prospective measurement or the retrospective measurement as the one or more inputs to a model of the one or more models.

At least one aspect is directed to a method. The method can include a data processing system receiving, from an application, a media of a person and data on health of the person. The method can include identifying, by one or more models of a data processing system, a presence of pain and a level of pain being expressed in the media of the person responsive to providing the media of the person and the data on health of the person as one or more inputs to the one or more models. The one or more models can be trained using a plurality of media and a plurality of data on health of persons expressing a plurality of levels of pain. The method can include generating, by the data processing system for the application, a notification identifying the presence of pain and the level of pain expressed by the person.

The method can include training, by the data processing system, the one or more models to identify the presence of pain using at least the plurality of media. The plurality of media can comprise at least one of a plurality of videos or a plurality of images of one or more body parts of a plurality of body parts of the plurality of persons expressing pain and not expressing pain. The method can include training, by the data processing system, the one or more models to identify the level of pain based at least on the plurality of data, wherein the plurality of data comprises vital sign data of the plurality of persons experiencing the plurality of levels of pain.

The method can include determining, using the one or more models, the level of pain of the person based at least on the data on health including at least one of a heart rate, a systolic blood pressure and age of the person input into the one or more models. The method can include determining, using the data on health input into a model of the one or more models comprising a neural network, at least one of the presence of pain or the level of pain. The data on health can comprise at least one of a medical history of the person or a measurement of a sensor attached to a body of the person.

The method can include identifying, by the data processing system from the received media, at least one of a video or an image of the received media depicting a portion of a face of the person. The method can include identifying, using the one or more models, at least one of the presence of pain or the level of pain responsive to providing the at least one of the video or the image as an input of the one or more inputs to the one or more models. The method can include receiving, by the data processing system, the media comprising a video capturing a movement of a plurality of parts of a body of the person. The method can include identifying, using the one or more models, at least one of the presence of pain or the level of pain responsive to the movement.

The method can include receiving, by the data processing system, the data on health comprising at least one of a prospective measurement or a retrospective measurement of a sensor of a wearable device of the person. The method can include identifying, by the data processing system at least one of the presence of pain or the level of pain responsive to providing the at least one of the prospective measurement or the retrospective measurement as the one or more inputs to a model of the one or more models.

At least one aspect is directed to a non-transitory computer-readable media having processor readable instructions, such that, when executed, cause at least one processor to receive, from an application, a media of a person and data on health of the person. The instructions, when executed, can cause the at least one processor to identify, using one or more models, a presence of pain and a level of pain being expressed in the media of the person responsive to providing the media of the person and the data on health of the person as one or more inputs to the one or more models. The one or more models can be trained using a plurality of media and a plurality of data on health of persons expressing a plurality of levels of pain. The instructions, when executed, can cause the at least one processor to generate, for the application, a notification identifying the presence of pain and the level of pain expressed by the person.

The instructions, when executed, can cause the at least one processor to train the one or more models to identify the presence of pain using at least the plurality of media. The plurality of media can comprise at least one of a plurality of videos or a plurality of images of one or more body parts of a plurality of body parts of the plurality of persons expressing pain and not expressing pain. The instructions, when executed, can cause the at least one processor to train the one or more models to identify the level of pain based at least on the plurality of data. The plurality of data comprises vital sign data of the plurality of persons experiencing the plurality of levels of pain.

The instructions, when executed, can cause the at least one processor to determine the level of pain of a plurality of levels of pain of the person based at least on the data on health including at least one biometric data point such as that of a heart rate, temperature, oxygen level (SpO₂), respiratory rate, a systolic blood pressure, and age of the person input into the one or more models. The instructions, when executed, can cause the at least one processor to determine, using the data on health input into a model of the one or more models comprising a neural network, at least one of the presence of pain or the level of pain. The data on health comprises at least one of a medical history of the person or a measurement of a sensor attached to a body of the person.

These and other aspects and implementations are discussed in detail below. The foregoing information and the following detailed description include illustrative examples of various aspects and implementations and provide an overview or framework for understanding the nature and character of the claimed aspects and implementations. The drawings provide illustration and a further understanding of the various aspects and implementations and are incorporated in and constitute a part of this specification. The foregoing information and the following detailed description and drawings include illustrative examples and should not be considered as limiting.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 depicts a block diagram of an example architecture of a computing system that can be used to implement one or more elements of the technical solutions described and illustrated herein.

FIG. 2 depicts an example of a system for training, deploying, implementing and using one or more AI or ML models for identifying and assessing pain.

FIG. 3 depicts an example of a pain assessment tool chart.

FIG. 4 depicts an example of a defense and veterans pain ratings scale.

FIG. 5 depicts an example of a table of results from an AI or ML model providing a classification of determinations of the presence of pain using the data.

FIG. 6 depicts an example of results of two AI or ML models for detecting the presence of pain.

FIG. 7 depicts an example chart of features of health data that can be used to predict pain levels experienced by a person.

FIG. 8 depicts an example of results or outputs of AI or ML models utilizing health data.

FIG. 9 depicts an example of three AI or ML models used to predict pain experienced by patients using one or more inputs.

FIG. 10 is a flow diagram of an example method of implementing a model for assessing pain of a patient using images and health data.

FIG. 11 depicts an example of a user device displaying a pain status output with the results of the pain assessment ML modeling on a graphical user interface.

FIG. 12 depicts an example of a graph of a pain trend of a patient over a course of a week, including a subjectively determined pain levels an objectively determined pain levels.

FIG. 13 depicts an example of images of a patient performing a gesture with multiple portions of the patient's body which the ML models can utilize to identify and assess pain.

FIG. 14 depicts an example of a graphical user interface showing an image of a person along with data determined by the ML models.

FIG. 15 depicts an example of a graphical user interface showing an image of a person along with data on pain location corresponding to the pain location on the person's body.

### DETAILED DESCRIPTION

Following below are more detailed descriptions of various concepts related to, and implementations of, methods, apparatuses, and systems of using a device executed application and AI or ML modeling to identifying presence of pain or pain levels experienced or expressed by a patient. The various concepts introduced above and discussed in greater detail below may be implemented in any of numerous ways.

The technical solutions of the present disclosure provide systems and methods for pain detection via ML applications, including for example, ML or AI based models for detecting, determining, detecting, assessing, quantifying or predicting pain experienced or exhibited by an injured person (e.g., a patient). A person, such as a patient, can experience different types of pain of varying levels or intensity. In addition, different patients react to and deal with pain differently. As a result, pain is often unreported, untreated and cause interference with person's duties, responsibilities or performance. In addition, as different patients can have different reaction or tolerance to pain, it can be challenging to accurately, consistently and objectively establish or quantify the level of pain experienced by the patients, even when the pain is reported. As a result, the medical professionals (e.g., doctors and nurses) find it challenging to accurately, consistently and objectively detect and monitor pain experienced by different patients.

The technical solutions of this solution overcome these challenges by using image capture technology, artificial intelligence (AI) or machine learning (ML) algorithms along with user device applications to accurately, consistently and objectively identify, detect, assess, quantify and report the level of pain experienced or exhibited by various patients. These solutions can utilize one or more ML models with image classification techniques to identify and quantify pain levels. The models can be trained using diverse datasets, including a national emergency medical services information system (NEMSIS) that encompasses data collected from emergency 911 calls related to head injuries. The ML models are trained to distinguish, detect, determine, predict, or assess the presence and intensity of pain, categorizing it into different levels. The architecture includes convolutional neural networks, image classifiers, and, as an example, a ResNet34 model. These models are trained for a specified number of epochs, and functions for data analysis are implemented to improve pain detection using inputs such as age, vital signs, and image data. The system allows for prospective or retrospective analysis of data, and the models can be applied to predict and verify reported pain levels. The application of the solutions extends to various populations, including adults, children, men, women, and military personnel, with the potential integration of military-specific health data.

The technical solutions can utilize AI or ML models for detecting and identifying pain levels using media, such as video fragments or images of any part of a patient's body, such as any combination of one or more of a person's face, eyes, fingers, hands, arms, legs, torso or any other part of a patient's body. ML models can be configured to monitor, analyze and detect or determine presence and intensity of pain experienced by a person, based on the motion of the person, such as a body movement (e.g., body language such as limping, holding onto an arm, a back or a torso). For instance, one or more ML models can achieve a diagnosis of up to 100% accuracy using agnostic image classification of pain with respect to a head injury. In such an ML model, data inputs, such as age, heart rate, and systolic blood pressure can be used and act as predictors or indicators of different levels of pain experienced by the person. For example, one or more ML models can determine whether or not the person is experiencing pain or for the persons experiencing pain, the ML models can determine the level of pain the person is experiencing, such as a high level of pain, a medium level of pain or a low level of pain. The one or more ML models of the present solution can be integrated into, communicatively coupled with or accessed by an application (e.g., a mobile application) that can include computer code and the functionality to execute or operate on a device (e.g., a mobile device or a tablet of a medical professional). The application can be configured to utilize a data processing system using or executing the one or more ML models, allowing for a user (e.g., a patient, a doctor, a nurse or any other medical professional) to individualize pain assessment based on various, prior, retrospective and prospective clinical data.

ML models can be trained to make determinations based on observations, analyses or information of any part of a body of a person, such as a person's face (e.g., facial expressions), eyes (e.g., movement, expansion or contraction pupils), shape of eyebrows or mouth, positioning or movement of back or shoulders, arms, legs, torso, or general body movements (e.g., hand gestures, type of walk, limping) or any other combination of body parts. ML models can be trained such that a single model analyzes, processes and makes determinations based on information about all of the body parts in combination. ML models can be trained such that each individual ML model focuses on a single body part. For instance, an ML model can be trained to analyze presence or level of pain based on facial expression. An ML model can be trained to analyze presence or level of pain based on hand gestures. Another ML model can be trained to analyze presence or level of pain based on body movements, body language or demeanor of a person. Another ML model can be trained to analyze presence or level of pain based on a combination of vital signs (e.g., heart rate or blood pressure). ML models can have their outcomes combined to produce a result (e.g., output determination of the presence or level of pain).

For example, high rates of pain can be encountered among active duty service members who have experienced various military service related injuries. In some instances, 63% of prevalence of pain can be reported among the active duty service members, leaving a large percentage of personnel experiencing pain underreported. Persons experiencing pain, including service members, can benefit from improved pain assessment capabilities.

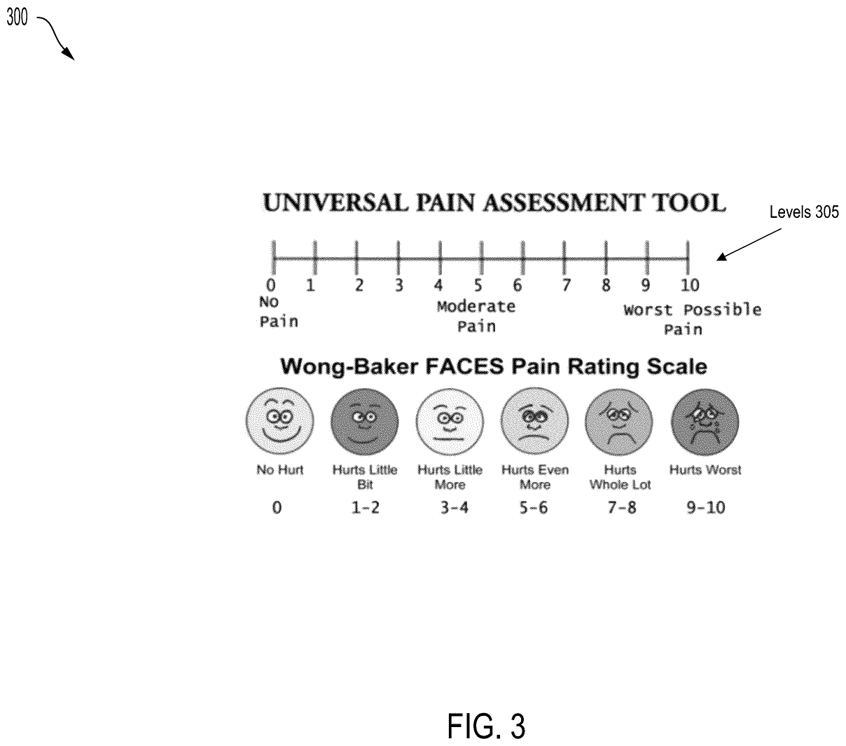

The techniques used by medical professionals to assess pain have evolved over time. By around 1960's, the pain was understood to be whatever the experiencing person says it was and existing whenever the experiencing person says it does. By about 1980's, the faces pain rating scale was used, such as the one shown in example 300 of FIG. 3 in which a universal pain assessment tool shows a scale of pain and six faces with descriptions of different pain levels for the patient to identify. Example 300 presents a scale of 10 levels 305 of pain. Each level 305 can correspond to a particular grade or level of pain as described or experienced by a patient, where level 305 of zero (0) corresponds to no pain, levels 305 of about 4-6 correspond to about a moderate level of pain and level 305 of 10 corresponds to the worst possible pain. Therefore, levels 305 can allow a patient or a person to provide a subjective assessment or quantification of the level of pain (e.g., pain level 305) experienced by the person.

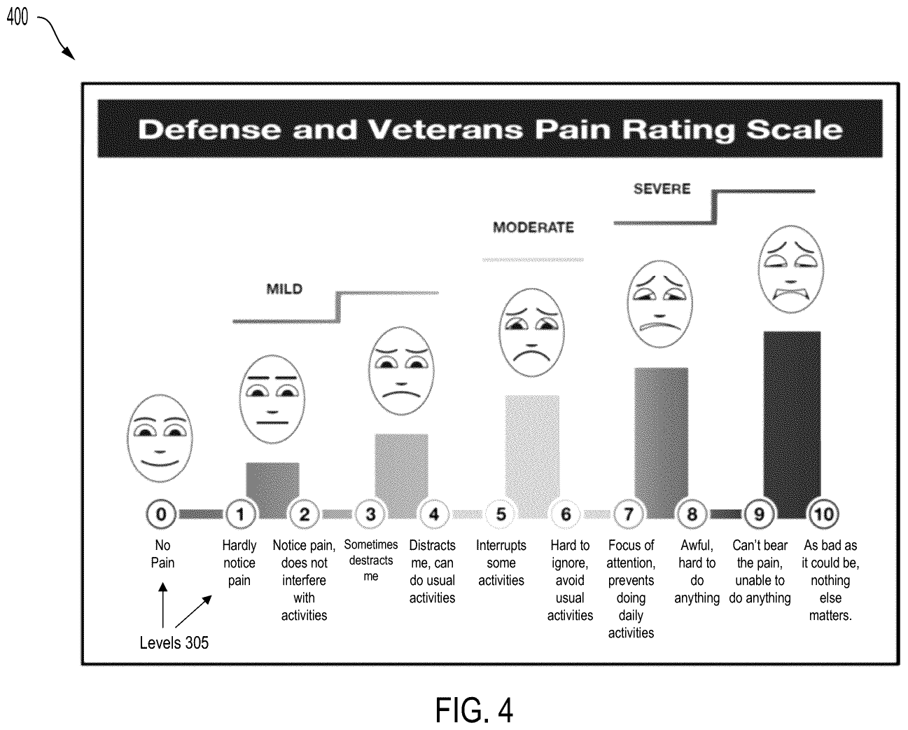

By about 2010's, in order to provide a more descriptive rating for assessing the pain, the defense and veterans pain rating scale was used, such as the one provided in example 400 of FIG. 4 in which a different chart with six faces and 10 different pain descriptions are provided for the patient to identify. In example 400, levels 305 correspond to ten different levels of pain, where pain level 305 of zero denotes no pain at all, level 305 of 1 corresponds to hardly noticeable pain, level 305 of 2 corresponds to noticed pain that does not interfere with activities, level 305 of 3 corresponds to pain that sometimes distracts, level 305 of 4 denotes a pain that distracts, but which is tolerable enough to do usual activities and level 305 of 5 denotes a pain that interrupts some of the activities. The same scale include a level 305 of 6 that corresponds to a pain level that makes it hard to ignore, leading to avoidance of usual activities, pain level 305 of 7 corresponds to a pain that is the focus of attention and prevents doing daily activities, pain level 305 of 8 corresponds to an awful pain making it hard to do anything, pain level 305 of 9 corresponds to a pain that a person cannot bear, making the person unable to do anything and pain level 305 of 10 correspond to a pain that is as bad as it could be, where nothing else matters. Therefore, example 400 provides a more detailed scale of 10 levels 305 of pain for the person to use to describe the pain they experience. However, in both examples 300 and 400, pain levels are identified by a person alone, making any such assessment subject to variations in terms of personal pain experience and tolerance, making it challenging to accurately, objectively and consistently assess the pain levels.

However, in order to provide a more accurate, consistent and uniform solution for detecting and assessing pain, the technical solutions can utilize any combination of an image capture technology, including AI or ML and Python-based functions to assess the presence, degree and likelihood of pain and self-reporting pain. The technical solutions can use one or more ML models utilizing image classification to detect and quantify pain. The technical solution can use a model with backend datasets to determine, assess or predict the levels of pain experienced or exhibited by the patient. The technical solutions can integrate the image classification and backend datasets for predicting or determining pain levels with one or more predictive inputs, such as age, heart rate and systolic blood pressure. The technical solutions can detect, assess, determine or predict the presence of pain and the level of pain experienced by a person based on the input data.

In one example, an AI or ML model can receive a data set of images of faces of people in order to assess or determine the presence or scale of the pain being experienced by the people in the images of the data set. In an example, a data set can include images of 161 different persons of various genders and ages, including males, females, adults and children. These images can represent input data from different patients whose pain assessment can be completed using the present solution. The images can include images of males and females, adults and children and people of various ethnicities, races and any other personal characteristics or features.

The AI or ML models can be trained using a data set (e.g., health data of patients) that can include a national emergency medical services information system (NEMSIS). The NEMSIS data set can include data collected from emergency 911 calls. The NEMSIS data can correspond to various persons with head injuries or head injury diagnosis, and can correspond to persons of all ages, ganders and races. The AI or ML models can be trained by the ML trainer to distinguish, detect, determine, predict or assess the presence or non-presence of pain. The AI or ML models can be trained by the ML trainer to distinguish, detect, determine, predict or assess different levels of pain experienced by a person. For example, the ML model can distinguish between low pain (e.g., pain levels 1-5) and high pain (e.g., pain levels 6-10), or can determine or identify any gradient level of pain, such as any level of pain from 0 through 10.

The technical solutions can utilize a balanced and split data. Balanced data can facilitate an equitable representation of different classes within a dataset, preventing bias in ML models for determining the presence or levels of pain. By addressing imbalances in data distribution, ML models can more effectively generalize (e.g., perform with a greater accuracy on new unseen data) across various pain levels. Data splitting, involving the division of datasets into training, validation, and testing sets, can be used to facilitate the evaluation of ML model performance on unseen data. This approach can improve an ML model's ability to make accurate predictions and avoids overfitting. For example, in a pain detection ML model, the combination of balanced data and data splitting strategies can facilitate development of more robust and reliable assessments and predictions (e.g., identification or detection) of pain levels.

The solutions can use convolutional neural network and an image classifier, such as an image classification model that is pre-trained using an image based data set. The model can include any number or combination of input layers, convolutional layers, fully connected layers, output layers and residual blocks. The model can utilize pooling techniques, such as global average pooling to provide average of feature maps as fixed-size output to be fed into fully connected layers. For example, the solution can utilize an image classification or vision task model, including for example a ResNet34 model architecture.

The solution can utilize one or more functions, including neural network related functions that can be implemented in or alongside one or more Python-based functions. The AI or ML models can be trained using the neural network for the data set for anywhere between 5 and 30 epochs, such as for example 15 epochs. For example, a random forest classifier can be used for NEMSIS data (or any other prospective and/or retrospective data) to determine feature importance of pain (6-10) versus low pain (1-5). In some implementations, a Python web App can use trained AI or ML models (e.g., pain presence model 230 and pain level model 235) which can be implemented on a local or a remote device or on a cloud. The application (e.g., a mobile application) can be a web application utilized via a mobile device to predict pain of a patient from one of the input images, based on the inputs entered via the application or a local device and potentially using randomly generated vital sign data.

Using the prospective or retrospective data (e.g., images from a data set) the one or more ML models can objectively detect, assess, determine or predict the pain and the level of pain experienced by a person. For instance, using the retrospective data sets, ML models can effectively predict and/or verify degree of reported pain. Data sets for both input and training of the ML models can be tailored to specific patients, such as adults, children, men, women or military personnel, including for example people with military injuries or people with military age groups.

One or more functions for data analysis can be implemented and utilized to improve and refine the detection and assessment of pain using health data inputs, such as age, vital signs, and image data. Data used can include, for example, diagnoses of strokes and heart attacks as well as other health data. Data from the Military Health System and Federal Interagency Traumatic Brain Injury Research (FITBIR) Information Systems can be included and used. FITBIR data can be explored to translate this process to military-specific populations.

The technical solutions can facilitate creation of a profile that is unique to a patient, based on the pain assessment or detection. For example, upon identifying or detecting the pain or assessing the pain level of a patient, the level of pain can be compared to that individual or compared to pain thresholds of others based on all the data. This can create a biosignature unique to the patient, but also showing how their rated pain corresponds to others' perceptions of pain. In doing so, a unique profile for a user can include unique aspects of that individual with respect to pain tolerance or expressions.

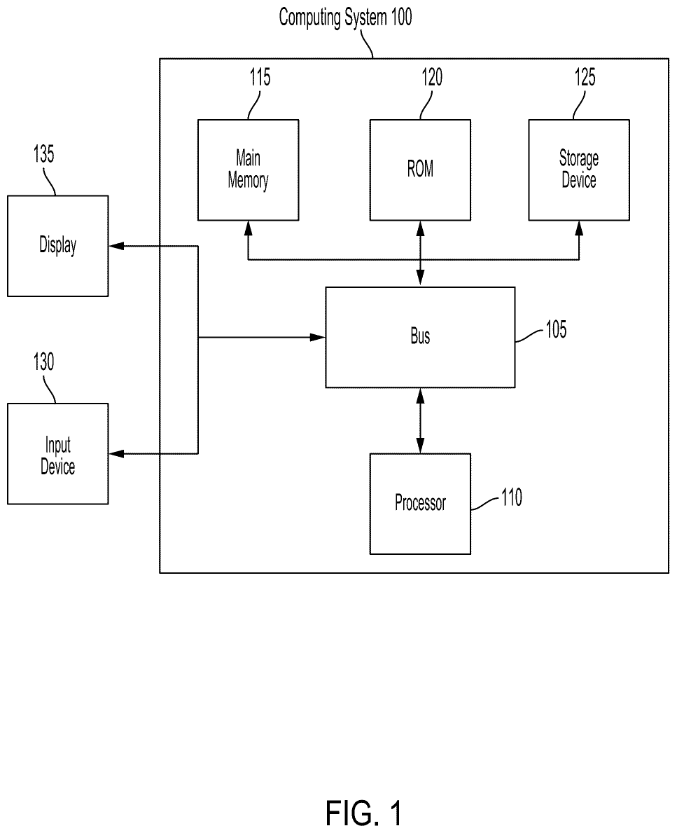

FIG. 1 depicts an example block diagram of an example computing system 100 that can be utilized in any computing device discussed herein, such as a server 205, mobile device 245 or wearable device 290 of a patient. The computing system 100 can include or be used to implement a ML model trainer 210 or its components (e.g., models 230 or 235) and to execute or use ML models, input and receive various data and communicate over a computer network 101. The computing system 100 includes at least one bus 105 or other communication component for transmitting or communicating information and at least one processor 110 or processing circuit coupled to the bus 105 for processing information. The computing system 100 can also include one or more processors 110 or processing circuits, such as processors, microcontrollers, one or more systems on a chip, any of which can be coupled to various components of the computing system 100. The computing system 100 also includes at least one main memory 115, such as a random access memory (RAM) or other dynamic storage device, coupled to the bus 105 for storing information, and instructions to be executed by the processor 110. The main memory 115 can be used for storing information during execution of instructions by the processor 110. The computing system 100 may further include at least one read only memory (ROM) 120 or other static storage device coupled to the bus 105 for storing static information and instructions for the processor 110. A storage device 125, such as a solid state device, magnetic disk or optical disk, can be coupled to the bus 105 to persistently store information, including data and instructions.

The computing system 100 may be coupled via the bus 105 to a display 135, such as a liquid crystal display, or active matrix display, for displaying information to a user such as a clinician or a doctor. An input device 130, such as a keyboard or voice interface, a camera, a microphone or a sensor may be coupled to the bus 105 for communicating information and commands to the processor 110. The input device 130 can include a touch screen display 135. The input device 130 can also include a cursor control, such as a mouse, a user touch screen interface function, a trackball, or cursor direction keys, for communicating direction information and command selections to the processor 110 and for controlling cursor movement on the display 135.

The processes, systems and methods described herein can be implemented by the computing system 100 in response to the processor 110 executing an arrangement of instructions contained in main memory 115. Such instructions can be read into main memory 115 from another computer-readable medium, such as the storage device 125. Execution of the arrangement of instructions contained in main memory 115 causes the computing system 100 to perform the illustrative processes described herein. One or more processors in a multi-processing arrangement may also be employed to execute the instructions contained in main memory 115. Hard-wired circuitry can be used in place of or in combination with software instructions together with the systems and methods described herein. Systems and methods described herein are not limited to any specific combination of hardware circuitry and software.

Although an example computing system 100 has been described in FIG. 1, the subject matter including the operations described in this specification can be implemented in other types of digital electronic circuitry, or in computer software, firmware, or hardware, including the structures disclosed in this specification and their structural equivalents, or in combinations of one or more of them. Likewise, computing system 100 can be implemented on a server, a cloud-based system or a cloud platform, a computer device, a mobile device, a wearable device, a system for implementing medical data measurements from sensors (e.g., vital sign data) or any other system for processing information.

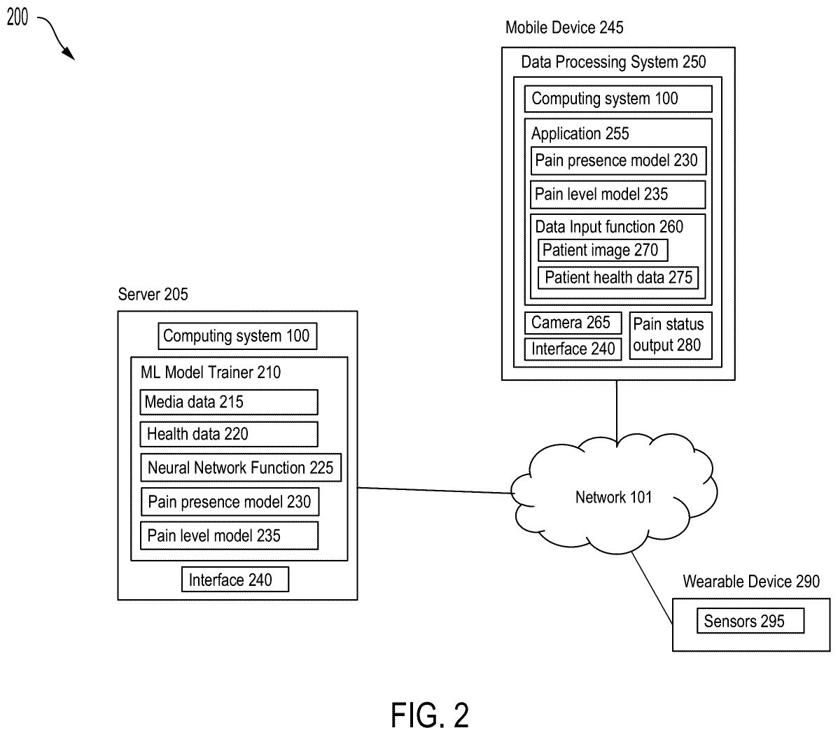

FIG. 2 depicts a block diagram of a system 200 for using one or more AI or ML models and applications to identify, detect, assess or determine the presence and the level of pain a person exhibits or experiences. System 200 can include one or more servers 205 communicating with one or more mobile devices 245 and one or more wearable devices 290, via one or more networks 101. A server 205 can include one or more computing systems 100, one or more ML model trainers 210 and one or more interfaces 240 for network communication. An ML model trainer 210 can include various datasets, such as one or more media data 215 and one or more health data 220 to be used with one or more neural network functions 225 to train one or more pain presence models 230 and pain level models 235. The wearable device 290 can be configured to, coupled with or in communication with a person or patient and can gather data (e.g., vital sign data measurements) of the patient using sensors 295.

Across the network 101, a device 245, also referred to as a mobile device 245, can include one or more data processing systems (DPS) 250. A DPS 250 can include one or more computing systems 100 executing one or more applications 255, operating and capturing media data 215 via one or more cameras 265, and using one or more interfaces 240 to communicate one or more pain status outputs 280. The application 255, also referred to as a mobile application 255, can include, have access to, or utilize one or more pain presence models 230, one or more pain level models 235 and one or more data input functions 260, each of which can include one or more patient images 270 and one or more patient health data 275. The patient images 270 or patient health data 275 can include measurements from sensors 295 on the wearable devices 290 of the patient, allowing the DPS 250 to utilize prospective or retrospective patient data as inputs and operation of the ML models 230 and 235.

At a high level, a server 205 that can utilize a computing system 100 to implement or execute an ML model trainer 210 to train one or more ML models for detecting presence of pain or level of pain experienced or exhibited by the patient, such as a pain presence model 230 and the pain level model 235 (e.g., collectively models 230 and 235). For example, an ML model trainer 210 on a server 205 can utilize media data 215 (e.g., images of patients or persons) and health data 220 (e.g., vital sign data from various sensor measurements, medical history or and other health information of a patient) to train the models 230 and 235. The ML models 230 and 235 can be trained using, for example, one or more neural network functions 225. Once the models 230 and 235 are trained, the server 205 can provide the trained models 230 and 235 to the device 245 via interfaces 240 of the server 205 and device 245. DPS 250 on the mobile device 245 can receive and utilize the models 230 and 235 (e.g., via the computing system 100) to provide outputs from these models to the user (e.g., a doctor or other medical professionals) via application 255. While allowing the user to access and use the ML models 230, application 255 can provide a data input function 260 to add the patient image 270 data (e.g., images of the patient) and/or patient health data 275 (e.g., vital sign data or other health related information of the user).

Media data 215 and health data 220 can be used to train pain presence model 230 and the pain level model 235. Data 215, 220, as well as patient image 270 and patient health data 275 can each be collected from various sensors 295, which can be deployed independently on wearable devices 290. Collected data can be provided to the DPS 250 via network 101. Patient image 270 and patient health data 275 can be input into the models 230 and 235. Models 230 and 235 can, based on the input information, provide a pain status output 280 which can include output of the models 230 or 235 indicating the presence (or absence) of pain, as well as levels of pain that the person is experiencing or exhibiting.

Wearable devices 290 can include any devices that a user can wear or carry, such as a smartwatch, a smartphone or head mounted device (HMD). Wearable devices 290 can include various sensors 295 for collecting any patient related data, such as vital sign data, such as heart rate (e.g., pulse) data, blood pressure, respiratory rate, body temperature, oxygen saturation (SpO2), blood glucose level, electrocardiogram (ECG or EKG) data, capnography, body weight, body composition, skin conductance, pulse wave velocity, temperature of extremities, cerebral oximetry, intracranial pressure or any other data or information about the patient.

Sensors 295 can include any devices (e.g., sensors or detectors) for capturing measurements or sensing signals. Sensor 295 can include any device or instrument that detects and measures physical, chemical, or biological properties and converts them into signals or data. Sensors 295 can be used to monitor patients' physiological parameters, such as, electrocardiogram (ECG) sensors for heart activity, photoplethysmogram (PPG) sensors for blood volume changes, blood pressure sensors, respiratory sensors for breath monitoring, temperature sensors, oxygen saturation sensors (pulse oximeters), glucose sensors for blood sugar levels, electroencephalogram (EEG) sensors for brain activity, bioimpedance sensors for body composition, inertial measurement unit (IMU) sensors for movement analysis, galvanic skin response (GSR) sensors for stress detection, weight sensors, Dexcom or continuous glucose monitoring (CGM) sensors, infrared sensors for temperature measurement, near-infrared spectroscopy (NIRS) sensors for cerebral oximetry, and intracranial pressure (ICP) sensors for monitoring pressure inside the skull.

Pain presence model 230 and pain level model 235 can include any ML or AI model for detecting, assessing, or determining the presence, absence or the level of pain experienced by a person (e.g., a patient). Models 230 and 235 can utilize any ML or AI technique or data for determining if a person is experiencing pain or for determining the level of pain the person is experiencing. For example, models 230 or 235 can include convolutional neural networks (CNNs) and deep neural networks (DNNs) that can be trained and used for image data analysis. ML models 230 or 235 can utilize neural network functionalities (e.g., biases and weights applied to various aspects or features of training data) to capture and evaluate visual cues, such as facial expressions indicative of pain or other body movements indicative of pain. ML models 230 or 235 can include functionality, such as random forests or gradient boosting, which can be used to combine predictions from multiple models, accommodating diverse data sources, including any patient health data 275, such as sensor measurements of vital signs (e.g., respiratory rate, heart rate, blood pressure and body temperature) or medical histories. Models 230 or 235 can utilize recurrent neural networks (RNNs), such as Long Short-Term Memory (LSTM) networks, to analyze time-series data of the patient health data 275, such as historical medical data or vital sign measurements over time. Support vector machines (SVM) can be used for both image and numerical data, classifying pain levels. Transfer learning, incorporating pre-trained models fine-tuned for pain detection tasks, can be used when labeled datasets for particular types of pain assessment are limited. Data augmentation techniques can be used to train datasets for improved model generalization. For example, models 230 and 235 can utilize an input image of a person in order to compare the features of the image with the learned or trained indicators developed by the ML model trainer 210 to recognize if the person is experiencing pain.

Pain presence model 230 can be any model for detecting presence or absence of pain that can be experienced by a person. Pain level model 235 can include any model for determining any level of pain that can be experienced by a person. Models 230 and 235 can be trained using media data 215, health data 220, or any combination of media data 215 and health data 220. Pain presence model 230, as well as pain level model 235, can use functions or classifiers to determine the gender, race, age, weight, height and any other features of a person. Models 230 or 235 can include image or data classifiers, such as for example, ResNet34. Models 230 and 235 can include any combination of hardware and software for determining or predicting presence of pain or pain levels for any particular cohort, such as adults, children, males, females, military personnel, police officers, firefighters or any other particular group of people that can experience or suffer from injury or pain.

Pain presence model 230 or pain level model 235 can each include machine learning scripts, code or sets of instructions or any other AI or ML related functionality described herein. Models 230 or 235 can include one or more Similarity and Pareto search functions, Bayesian optimization functions, neural network-based functions or any other optimization functions or approaches. Models 230 or 235 can each include an artificial neural network (ANN) function or a model, including any mathematical model composed of several interconnected processing neurons as units. The neurons and their connections can be trained with data, such as any input data discussed herein. The neurons and their connections can represent the relations between inputs and outputs. Inputs and outputs can be represented with or without the knowledge of the exact information of the system model. For example, models 230 or 235 can be trained by model trainer 210 using neuron by neuron (NBN) algorithm.

Media or media data 215 can include any number of media, such as video files, audio files, images, document files, graphics files, 3D images or any other files for patients or persons that can be used as data sets (e.g., labeled data) for training ML models 230 or 235. For instance, media data 215 can include images and/or videos of faces and/or bodies of persons experiencing pain. For example, media data 215 can include hundreds, thousands or tens of thousands of videos (e.g., video fragments or files) or images of different persons experiencing one or more types of pain, such as headache or pain from an injury of head, back pain or pain from injury to the back, shoulder pain, eye pain, ear pain, toothache, pain from injuries to the limbs or any other type of pain. The images of faces can depict, include or relate to the same person and/or one or more people. Multiple AI or ML models can be developed to create a unique model for the individual person based on their own features and/or compared to other people in the data set.

Health data 220 can include any health-related data of persons that can be used as data sets (e.g., labeled data) for training ML models 230 or 235. Health data 220 can include information on age, heart rate, systolic blood pressure, information on stroke, heart attacks or any vital sign data pertaining to a patient. Health data 220 can provide information that can be correlated by the models 230 and 235 to predict levels of pain experienced by patients. Health data 220 can include NEMSIS data or any other medical history data corresponding to any number of patients or persons. Health data 220 can also include video based raw or processed signal data such as data received via photoplethysmogram (PPG) sensors 295 in a wearable device 290, such as sensors measuring heart rate, pulse oximetry, blood pressure, respiratory rate, stress, autonomic nervous activity, activity or motion of a person or any other information or data related to a person (e.g., a patient).

Patient image 270, also referred to as patient media data or patient metadata 270, can include any media (e.g., video, audio or image) of a patient or person whose pain is being detected or processed using ML modeling. Patient media data 270 can be captured for the patient for whom ML models 230 and 235 are used to determine the objective assessment of the pain level, using patient media data 270 as inputs to the ML models. Patient image 270 be captured by a camera 265 and can include one or more images of a patient, such as for example images similar to those in the media data 215. Camera 265 can include any camera for capturing media data (e.g., patient image 270 or media data 215. Cameras 265 can be deployed around a hospital or within a medical environment to capture images of patients. Cameras 265 can be integrated into or coupled with wearable devices 290, devices 245 or servers 205. Cameras 265 can include Patient image 270 can be input into the trained models 230 or 235 to determine the presence or absence of pain experienced by the patient (or pain level experienced by the patient). Cameras 265 can include any device for capturing visual information through optical sensors to generate digital images or videos, including specialized cameras, such as surveillance cameras for security, action cameras for dynamic activities, and medical imaging cameras for diagnostic purposes. Cameras 265 can include features like high resolution, low-light capabilities, and advanced image processing.

Patient health data 275 can include one or health information of a patient, such as health information stored in health data 220. Patient health data 275 can include any data or information on the patient or person whose pain is being detected. Patient health data 275 can be input into the trained models 230 or 235 to determine the level of pain experienced by the patient. Patient health data 275 can include medical history of a patient, patient's name, age, race, weight, height, medical conditions or details about medical treatments. Patient health data 275 can include measurements from sensors 295 of the wearable devices 290 of the person or patient. Patient health data 275 can include vital sign data of the person, or any other medical information that can be informative or correspond to pain experienced or exhibited by the person.

Data processing system (DPS) 250 can include any combination of hardware and software for implementing a application 255 and models 230 and 235. DPS 250 can include a processor, a controller, a microcontroller or a control circuit and can operate, execute, or be implemented by a computer system 100. DPS 250 can include functions, computer code, scripts or instructions stored in memory, such as memory 115 or storage 125, and can be executed on one or more processors, such as processor 110. DPS 250 can use data corresponding to patients or injured personnel, including injured military service members, in order to use them as inputs into the models 230 and 235.

Model trainer 210 can include any combination of hardware and software, such as scripts, functions and computer code stored in memory or operating on a processor for training models 230 or 235 and any of their functions or functionalities. Model trainer 210 can include the functionality to access and utilize media data 215 and health data 220 for a plurality of persons to train models 230 or 235 using neural network functions 225 or other ML methodologies. Model trainer 210 can perform the training using any number of artificial intelligence (“AI”) or machine learning (“ML”) functions or techniques. For example, model trainer 210 can include any combination of supervised learning, unsupervised learning, or reinforcement learning. Model trainer 210 can include the functionality including or corresponding to linear regression, logistic regression, a decision tree, support vector machine, Naïve Bayes, k-nearest neighbor, k-means, random forest, dimensionality reduction function, or gradient boosting functions. Model trainer 210 can include the functionality to perform neural network (e.g., DNN, RNN, CNN or ANN) learning of ML models 230 or 235, including generation or adjustment of any offsets or weights pertaining to any features of the training data set to adjust the ML model functionality.

Neural network function 225 can include any combination of hardware and software facilitating a neural network functionality to ML models 230 and 235 or otherwise providing an implementation of one or more neural networks. Neural network function 225 can include a computational model designed according to the structure and functioning of the human brain, designed to recognize patterns to make. Neural network function 225 can include interconnected nodes or artificial neurons, organized into layers. In a neural network, information can be processed through different layers, where each connection between nodes can be associated with a weight and adjusted by offsets which can have their values determined during training of the ML models. Neural network function 225 can facilitate training of the ML models or be included in a ML model. The network of the neural network model can learn from labeled samples (e.g., labeled media or health data), refining the weights or offsets to minimize the difference between predicted and actual outputs. Neural networks can be trained for identify, detect, assess, predict or recognize presence or levels of pain based on, for example, image recognitions, recognitions of movements, language processing (e.g., of health data or audio recordings), including any combination of data of the patient. Neural network function 225 can include any number of layers, including numerous hidden layers (e.g., deep neural networks) to provide or facilitate DNN modeling.

Network interface 240 can include any combination of hardware and software for communicating via a network 101. Network interface 240 can include scripts, functions and computer code stored in memory and executed or operating on one or more processors to implement any network interfacing, such as network communication via a network 101. Network 101 can include any wired or wireless network, a communication cable or a cable for transmitting information or data, a World Wide Web, a local area network, a wide area network, a Wi-Fi network, a Bluetooth network or any other communication network or platform. Network interface 240 can include functionality for communicating, via network 101, using any network communication protocol such as Transmission Control Protocol (TCP)/Internet Protocol (IP), user datagram protocol (UDP), or any other communication protocol used for communicating over a network 101. Network interface 240 can include communication ports and hardware for receiving and sending data and messages over the network 101 or via a power cable. Network interface 240 can include the functionality to encode and decode, send and receive any information, such models 230 and 235. Interface 240 can include the functionality to provide graphical user interface for a user to interface or communicate with a device (e.g., 245, 205 or 290), including for example menus for user selection and data inputs and for displaying notifications to the user.

Pain status output 280 can include any indication or output corresponding to the pain experienced, manifested or exhibited by the person. Pain status output 280 can include a notification or an indication of the presence or non-presence of pain with respect to a particular patient. Pain status output 280 can include a notification of the level of pain experienced by a user, such as a level of pain between 1-10, or any other scale or gradient. Pain status output 280 can be displayed to a display of a mobile device 245, server 205 or wearable device 290 of a user. Pain status output 280 can include the video or image data that can be integrated into a portable document format (PDF) that can be generated by the application 255 to offer image-based (e.g., rich media) data for review by a clinician or other medical professionals. The image data can identify or show the patient or indicate the location of the pain.

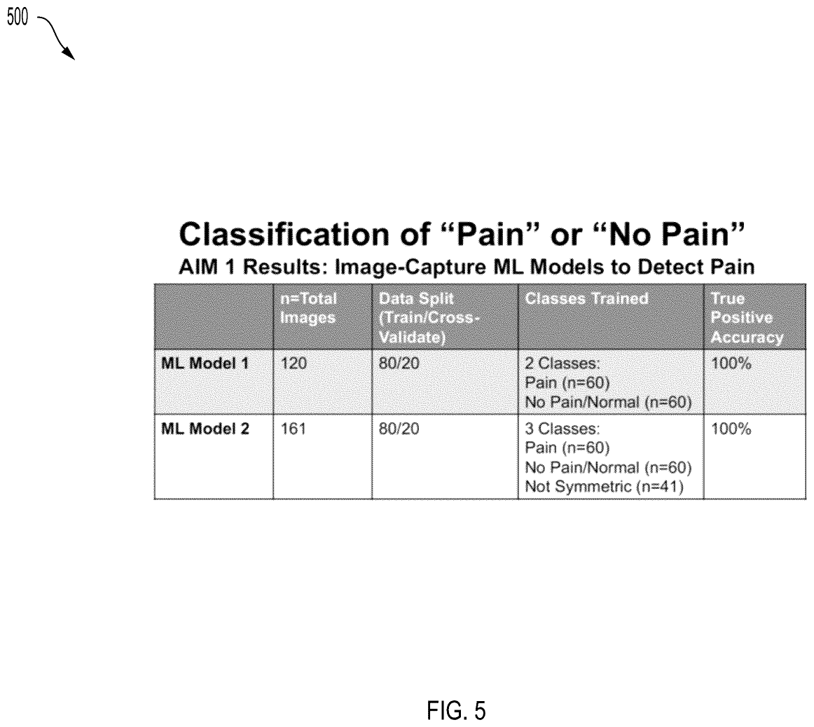

FIG. 5 depicts an example of a table 500 providing results of a test for classifying presence of data by two models, ML model 1 and ML model 2. ML models 1 and 2 can be, for example, a pain presence model 230 or a pain level 235. With respect to ML model 1, the ML model 1 can be trained using 120 image data set and can have data split of 80/20 with respect to train/cross-validate results. Classes trained for ML model 1 can include the presence of pain result for a data set of n=60 and absence of pain result (e.g., no pain or normal) for n=60, with the accuracy of detection of 100%. With respect to ML model 2, the ML model 2 can be trained using 161 image data set and can have data split of 80/20 with respect to train/cross-validate results. Classes trained for ML model 2 can include the presence of pain result for n=60, absence of pain result (e.g., no pain or normal) for n=60, and not symmetric (n=41). The accuracy of detection for ML Model 2 can be 100%.

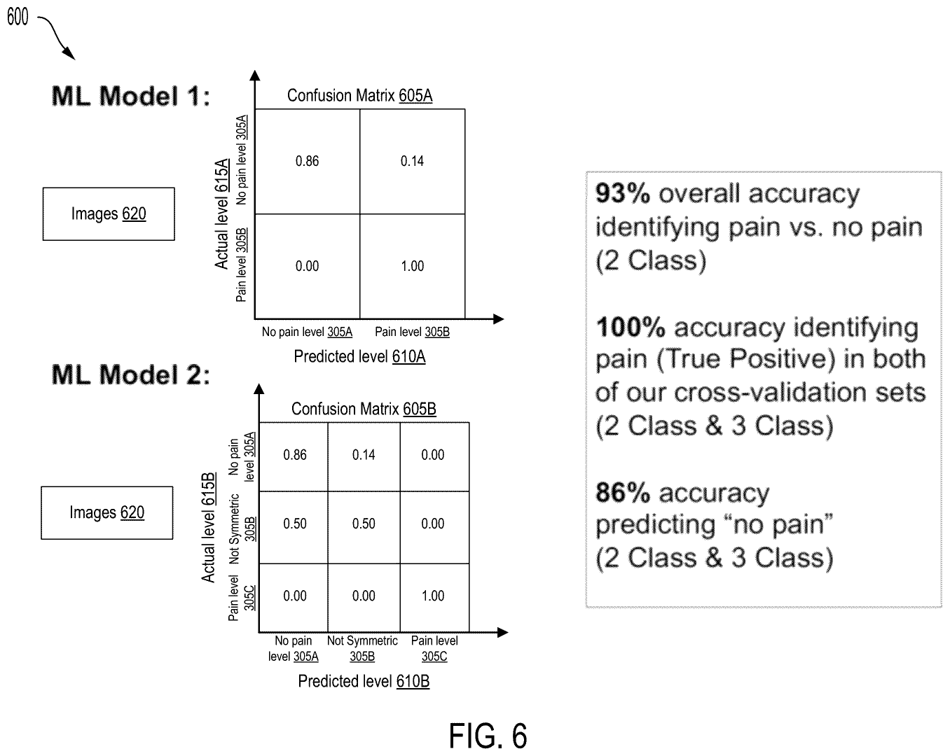

FIG. 6 depicts an example of a result 600 of a test of two models, ML model 1 and ML model 2 along with their confusion matrices illustrating actual and predicted pain detection outcomes. ML models 1 and 2 can both be, for example, a pain presence model 230 or a pain level 235. ML models 1 and 2 provided 93% overall accuracy identifying pain vs. no pain, 100% accuracy identifying pain (true positive) in both of the cross-validation sets and 86% accuracy of predicting absence of pain (e.g., no pain).

ML model 1 includes a confusion matrix 605A that corresponds to a dataset of images 620. Images 620 can include any number of images of any number of persons for training a ML model 1 (e.g., ML model 230 or 235 for determining presence or level of pain), including media data 220. Images 620 can include images of persons' faces, shoulders, hands, arms, legs, eyes or any other part of the body. Images 620 can include images or videos (e.g., video frames) and can represent gestures, positions or movements (e.g., via multiple video frames).

Confusion matrix 605A can include an x-axis of predicted level 610A corresponding to the ML model determined or predicted pain level, including a predicted no pain level 305A or a predicted pain level 305B. Confusion matrix 605A can include a y-axis of an actual level 615A corresponding to the actual level of pain, including an actual no pain level 305A and an actual pain level 305B. Confusion matrix 605A can include four quadrants. The first quadrant can correspond to an actual no pain level 305B and a predicted no pain level 305A, having a value of 0.86, which correspond to 86% of correlation or agreement between the actual and predicted no pain levels 305. The second quadrant can correspond to an actual no pain level 305A and a predicted pain level 305B, having a value of 0.14, which can correspond to 14% of agreement between these outcomes. The third quadrant can correspond to an actual pain level 305B and a predicted no pain level 305A, having a value of 0.00, which can correspond to 0.0% of agreement between these outcomes. The fourth quadrant can correspond to an actual pain level 305B and a predicted pain level 305B, having a value of 1.00 which can correspond to 100% of agreement between these outcomes.

Example 600 also includes a confusion matrix 605B, which can include an x-axis of a predicted level 610B corresponding to the ML model determined or predicted pain level, including a predicted no pain level 305A, not symmetric outcome 305B (e.g., undetermined pain level) and a pain level of 305C. Confusion matrix 605B can include a y-axis of an actual level 615B corresponding to the actual level of pain, including an actual no pain level 305A, not symmetric 305B outcome and an actual pain level 305C. Not symmetric outcomes 305B can correspond to, for example, determinations that are in between the presence and no presence of pain, and can be excluded from determinations in some implementations.

Confusion matrix 605A can include nine quadrants. In the first row, the first quadrant can correspond to an actual no pain level 305A and a predicted no pain level 305A, having a value of 0.86. The second quadrant can correspond to an actual no pain level 305A and a predicted not symmetric outcome 305B and can have a value of 0.14. The third quadrant can correspond to an actual no pain level 305A and a predicted pain level 305C and can have a value of 0.00. In the second row, the fourth quadrant can correspond to an actual not symmetric 305B outcome with a determined no pain level 305A of 0.50 value, while a fifth quadrant can correspond to an actual not symmetric 305B with a predicted not symmetric 305B determination of a value of 0.50, and sixth quadrant having an actual not symmetric 305B outcome and a determined pain level 305C of 0.00 value. In the third row, a seventh quadrant can include an actual pain level 305C with a no pain level determination of 0.00, an eight quadrant can include an actual pain level 305 with a not symmetric 305B determination of 0.00, while a ninth quadrant of an actual pain level 305C and a determined pain level 305C includes a value of 1.00 (e.g., 100%).

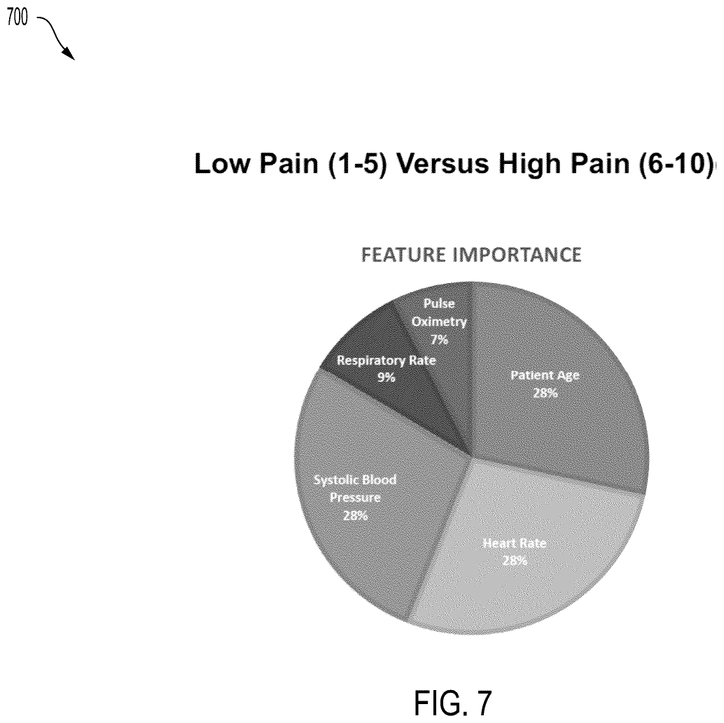

FIG. 7 depicts an example of a result 700 of features that can be beneficial in predicting or detecting different levels of pain (e.g., pain level model 235). For example, patient health data 275 can include patient age, heart rate and systolic blood pressure can be most beneficial at about 28% each, along with respiratory rate at 9% and pulse oximetry at 7%. These information or data can be included as a part of the health data 220 or patient health data 275 and can be used for training models 230 and 235 or as patient's own data to be input into the models 230 and 235 (e.g., in the event of the patient data).

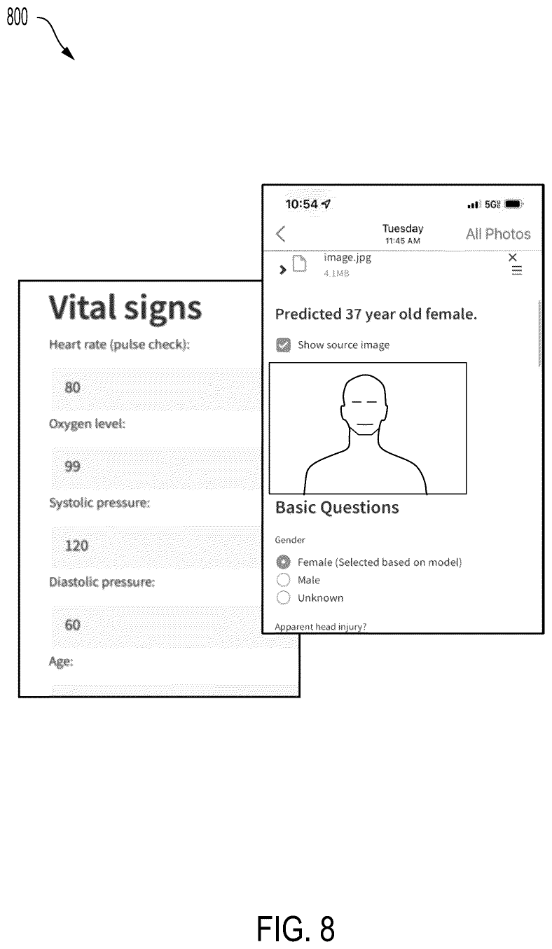

FIG. 8 depicts an example of a results 800 of a demo that can utilize both pain presence model 230 and pain level model 235. The functional demo can provide feature importance, such as particular vital signs data used for a determination. For example, the vital signs can include heart rate (e.g., pulse check) of about 80, oxygen level of 99, systolic pressure of 120, diastolic pressure of 160 and other patient health data 275, such as predicted age of the patient, gender of the patient and more. The features of the health data can include various degrees of importance in accurately detecting or determining presence or level of pain the patient experiences.

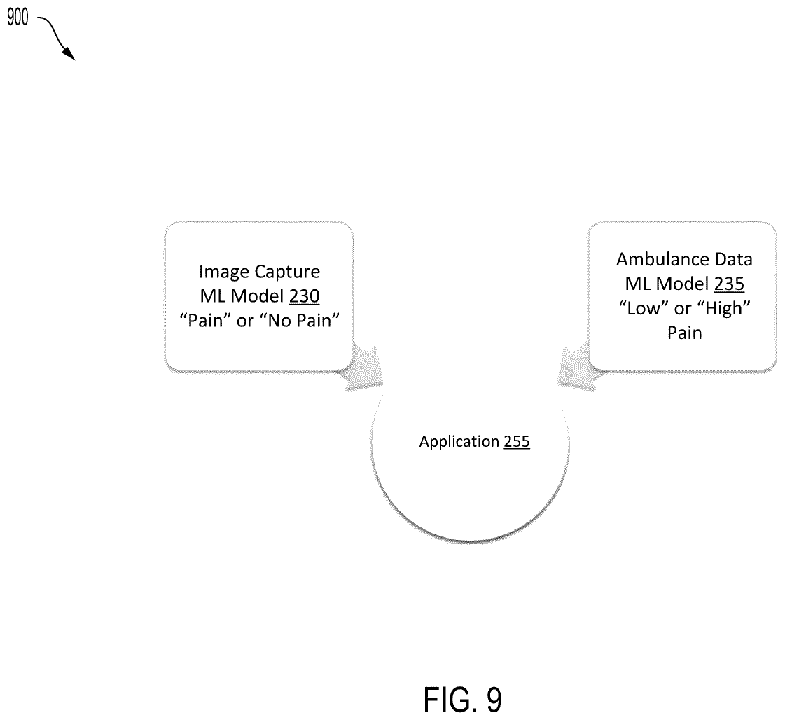

FIG. 9 depicts an example of a block diagram 800 of interactions between the AI model 1 (AIM 1), AI model 2 (AIM 2) and AI model 3 (AIM 3). AI models can correspond to any ML models 230 or 235 and vice versa. AI Model 1 can be a pain presence model 230 that can use the ML model functionality to determine the presence of pain (e.g., pain or no pain). AI model 2 can be a pain level model 235 that can utilize the ML model functionality to determine the level of pain experienced by the patient (e.g., high versus low pain levels). AI model 3 can include a model that can receive user inputs, such as the image capture and a classifier to determine the age, gender and other features of the user, as well as patient data inputs (e.g., image and health data). AI model 3 can then utilize AI models 1 and 2 functionalities to use the input data and run the models 230 and 235 and determine the pain status of the patient (e.g., pain presence and pain level).

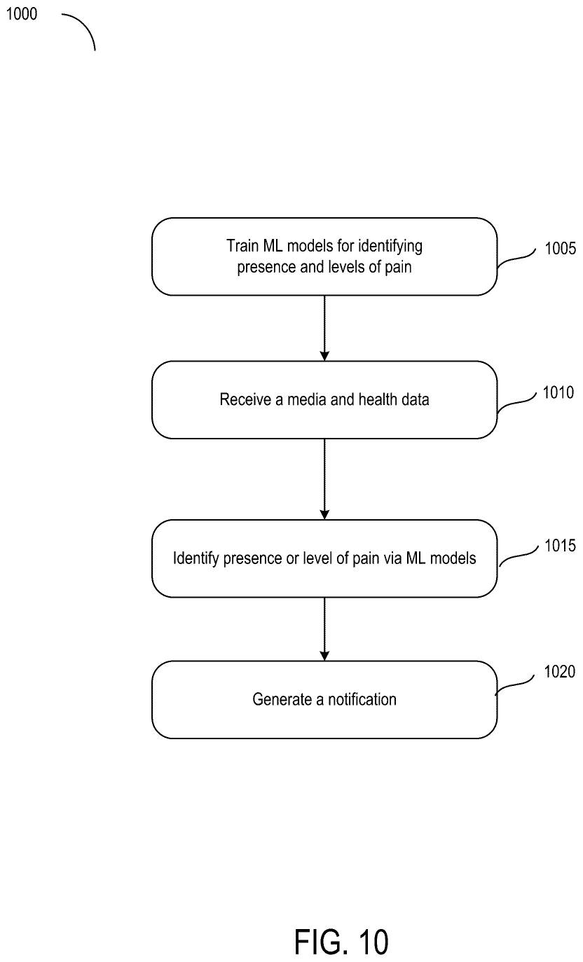

FIG. 10 depicts an example flow diagram of a method 1000 of identifying, assessing, quantifying, detecting or determining the pain experienced by a patient using machine learning and an application. Method 1000 can be implemented, for example, using example systems 100 and 200, along with any functionality of features described in connection with FIGS. 1-9 and 11-15. Method 1000 can include acts 1005-1020. At act 1005, the method can include training ML models for identifying the presence and levels of pain. At act 1010, the method can include receiving media and health data of a patient. At act 1015, the method can include identifying the presence or level of pain using ML models. At act 1020, the method can include generating a notification with the identified presence of pain or the level of pain of the patient.

At 1005, the method can include training ML models for identifying the presence and levels of pain. The method can include training one or more machine learning (ML) models to identify, assess, determine, quantify, detect or distinguish between the presence or non-presence of pain experienced or exhibited by a person (e.g., a patient). The method can include training one or more ML models to identify, assess, determine, quantify, detect or distinguish a level of pain experienced or exhibited by the person out of a plurality (e.g., two, three, four, five, 10 or more than 10) levels of pain that a person can experience.

A data processing system can utilize a ML model trainer to train the one or more models to detect or predict the presence or level of pain. The trainer can train the ML models using data sets that can include any number of media data or health data of any number of persons that may experience no pain or experience any level of pain. The media data can include images, videos, audio recordings, illustrations or depictions of persons experiencing no pain or varying amount of pain. The health data can include any information or data of persons or patients, including medical history, sensor readings (e.g., The ML models can be neural network models, such as RNN, DNN, CNN or any other neural network model. The trainer can train ML models to include offsets and weights corresponding to particular features within the training datasets providing distinctions indicative of pain presence, absence or non-presence of pain or levels of pain.

The method can include the trainer of the data processing system training the one or more models to identify the presence or manifestation of pain using at least the plurality of media. For example, a dataset of media (e.g., videos, images, audio, illustrations) can be used to train a pain presence model or a pain level model. The plurality of media can include, for example, at least one of a plurality of videos or a plurality of images of one or more body parts of a plurality of body parts of the plurality of persons expressing pain and not expressing pain. For instance, the plurality of videos can include or indicate gestures or movements, such as limping walk, self-soothing actions or gestures, trembling actions, or any other actions or movements for identifying presence or level of pain. For example, images of faces of persons can be used to indicate presence or level of pain. For example, images of eyes, including sizes of pupils or presence of redness in the eyes can be used to determine presence or level of pain.

The method can include a trainer of the data processing system training the one or more models to identify the level of pain based at least on the plurality of data on health. The plurality of data on health can include vital sign data of the plurality of persons experiencing the plurality of levels of pain. The vital sign data can include, for example, signals or measurements from sensors pertaining any health related information or state of the health of the person, such as heart rate (e.g., pulse), blood pressure, respiratory rate, body temperature, oxygen saturation, blood glucose level, body weight, capnography (e.g., concentration of carbon dioxide in exhaled breath), electrocardiogram (e.g., ECG or EKG data), pulse wave velocity (e.g., marker of arterial stiffness or cardiovascular health), cerebral oximetry or intracranial pressure (e.g., ICP).

At 1010, the method can include receiving media and health data of a patient. The method can include the data processing system, receiving, from an application executed on a device, at least one of a media of a person or a data on health of the person. For example, an application can execute on a smartphone, tablet or a computer of a medical professional (e.g., doctor, nurse or a medical technician). The application can have access to one or more ML models for detecting, assessing, identifying or quantifying presence of pain or level of pain of the patients. The application can receive information or data on a particular patient, including media data (e.g., video, image, audio or illustration) corresponding to the patient. The application can receive patient health data, including, for example, vital sign data of the patient, medical history of the patient or any other medical or health related information of the patient.

The method can include the data processing system receiving the patient image data or the patient health data from one or more cameras or sensors. The cameras can be deployed in a medical institution (e.g., a hospital, a clinic or a medical center). The sensors can provide measurements of vital sign data or other information from a wearable device coupled with, attached to or worn by the patient. The data processing system can receive the media of the person and the data on health of the person from the application on the mobile device. The patient image data and the patient health data can be a prospective data or retrospective data. The data can include, for example, a real-time stream of data on health of the patient. The data can include at least one of a prospective measurement or a retrospective measurement of a sensor of a wearable device of the person.

At 1015, the method can include identifying the presence or level of pain using ML models. The method can include the one or more models of the data processing system. The one or more models can identify a presence of pain or a level of pain. The presence of pain or the level of pain can be expressed in the media of the person responsive to providing the media of the person as one or more inputs to the one or more models. The presence of pain or the level of pain can be expressed responsive to providing the data on health of the person as one or more inputs to the one or more models. The one or more models can be trained using a plurality of media and a plurality of data on health of persons expressing a plurality of levels of pain.

The one or more models can determine the level of pain of the person based at least on the data on health including at least one of a heart rate, a systolic blood pressure and age of the person input into the one or more models. The data processing system can determine at least one of the presence of pain or the level of pain. Such a determination can be made using the data on health input into a model of the one or more models comprising a neural network. The neural network model can include any neural network model, such as a RNN, CNN or DNN model. The data on health can include at least one of a medical history of the person or a measurement of a sensor attached to a body of the person.

For instance, the method can include the data processing system identifying, from the received media, at least one of a video or an image of the received media depicting a portion of a face of the person. For instance, the method can include the data processing system identifying, using the one or more ML models, at least one of the presence of pain or the level of pain responsive to providing the at least one of the video or the image as an input of the one or more inputs to the one or more models.

The method can include the data processing system receiving the media comprising a video capturing a movement of parts of a body of the person and identifying, using the one or more ML models, at least one of the presence of pain or the level of pain responsive to the movement. For example, the data processing system can identify at least one of the presence of pain or the level of pain responsive to providing the at least one of the prospective measurement or the retrospective measurement as the one or more inputs to a model of the one or more models.

At 1020, the method can include generating a notification with the identified presence of pain or the level of pain of the patient. The method can include the data processing system generating, for the application, a notification identifying the presence of pain and the level of pain expressed by the person. In some implementations, the application can generate the notification. The notification can include the determination, identification, assessment, finding, or detection of the presence of pain, non-presence of pain or the level of pain the patient experiences, expresses or exhibits. For example, the notification can include a depiction of a silhouette, illustration or a graphic of a patient's body and an indication (e.g., highlighting or marking) of a portion of the body of the patient at which the pain is present or experienced. For example, the notification can include or indicate determinations of the model, such as the size of the pupil of the patient, the presence of redness or blood, description of the facial expression or a gesture or movement of the patient. The notification can be illustrated in a graphical user interface of the application on a user's device, such as a mobile device of the medical professional (e.g., doctor, nurse or a medical technician).

The notification can be output in a form of a pain status output on a graphical user interface. The notification can include the video or image data that can be integrated into a PDF file or a report for the user (e.g., clinician or a doctor). The PDF report can be generated by the application to offer image-based (e.g., rich media) data for review by a clinician or other medical professionals. The data in the graphical user interface can include or identify pain location, severity of pain (e.g., pain level), vital sign data, measurements of user's pupil sizes The image data can identify or show the patient or indicate the location of the pain.

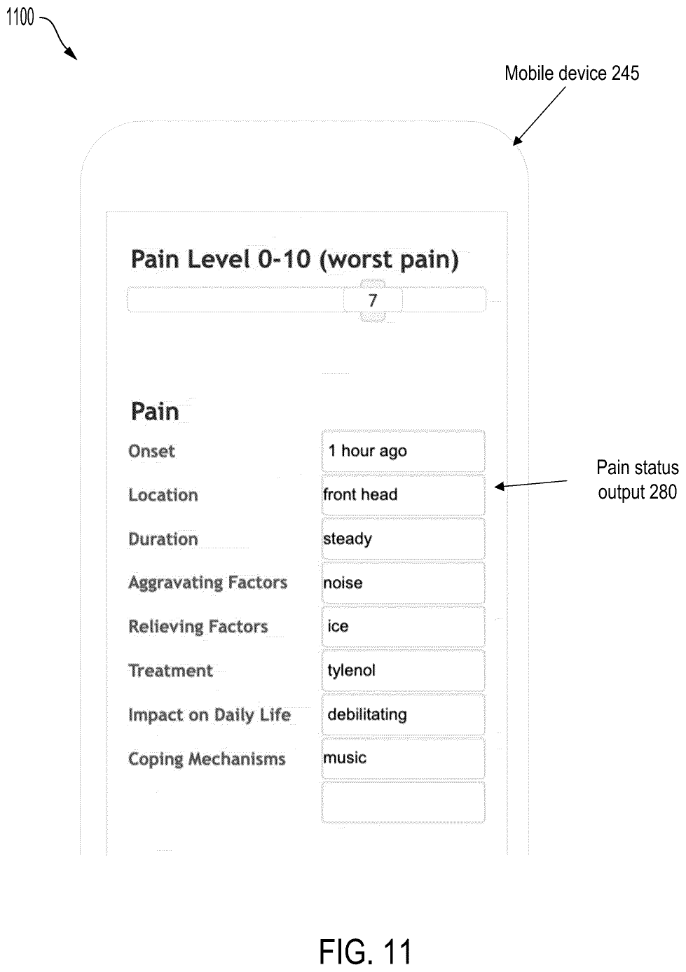

FIG. 11 depicts an example of a display of a pain status output 280 on a graphical user interface of a mobile device 245. The graphical user interface can be provided by an interface 240 of the mobile device 245. Graphical user interface can provide a display of a pain level 305 for the patient (e.g., person) ranked from 0-10, such as for example 7. The pain level 305 can be determined by the ML models, based on the patient's media data and personal health data input into the one or more ML models 230 or 235.

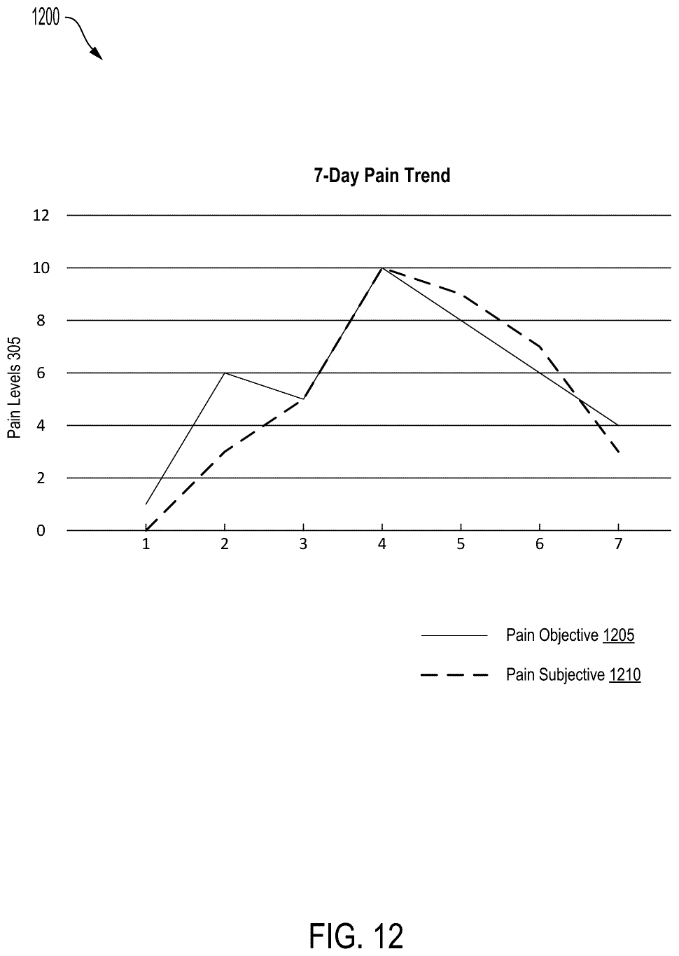

FIG. 12 depicts an example of a graph 1200 of a 7-day pain trend of a patient. The graph 1200 includes a full line denoting an objective pain determination by ML models (e.g., pain objective 1205) and a subjective pain determination by the patient shown as a dotted line of pain subjective 1210. Graph 1200 shows that on day 1, the pain objective 1205 begins at pain level 305 of 1, on day 2 the pain level 305 increases to 6, on day 3 the pain level 305 is at 5, on day 4 the pain level 305 is at 10, on day 5 the pain level 305 is at 8, on day 6 the pain level 305 is at 6, and on day 7 the pain level 305 is at 4. With respect to the pain subjective 1210, at day 1 the pain level 305 is at 0, at day 2 the pain level 305 is at 3, on day 3 the pain level 305 is at 5, at day 4 the pain level is at 10, on day 5 the pain level 305 is at 9, on day 6 the pain level is at 7, and on day 7 the pain level 305 is at 3. Accordingly, based on the graph 1200, the pain objective 1205 and pain subjective 1210 determinations track similar pattern of pain over the 7 day period.

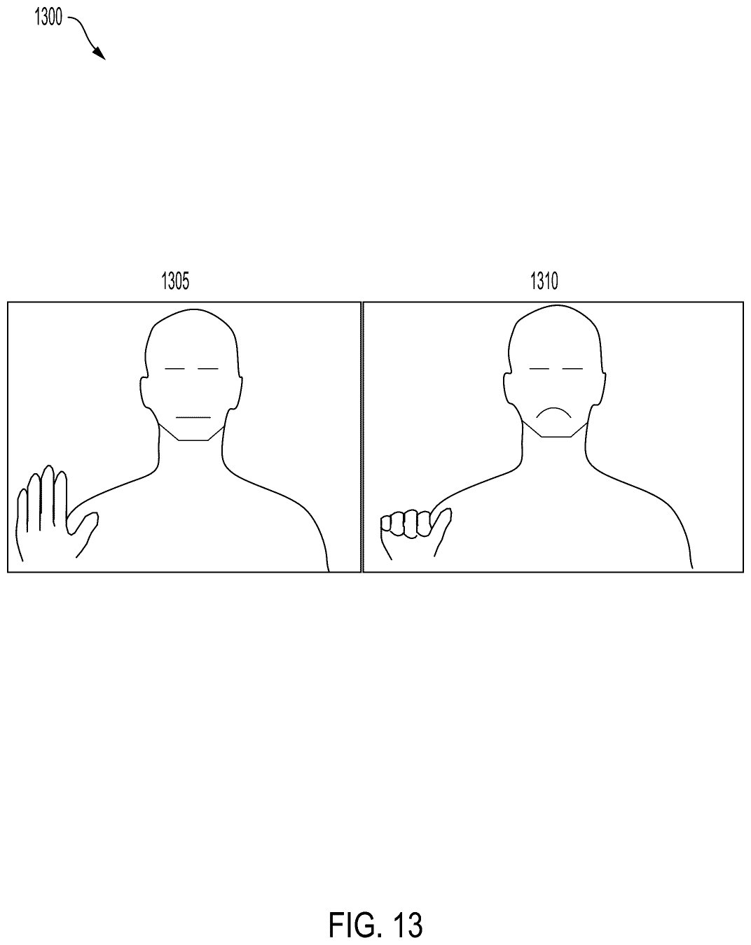

FIG. 13 depicts an example 1300 of images 1305 and 1310 of a person (e.g., patient) performing a gesture with a right hand and making a facial expression of pain. Images 1305 and 1310 can show multiple body parts of the person, such as a hand, a face, shoulders, neck, eyes and other parts which can be analyzed by the ML models in combination to determine the presence and the level of pain. For example, images 1305 can show a person's body parts other than just facial recognition to demonstrate how AI or ML models can detect and track pain associated with body parts other than the head. In image 1305, a person can show an open hand. At image 1310, the same person can show a closed hand and a painful act or expression, which can indicate a pain associated with arthritis or a finger fracture.

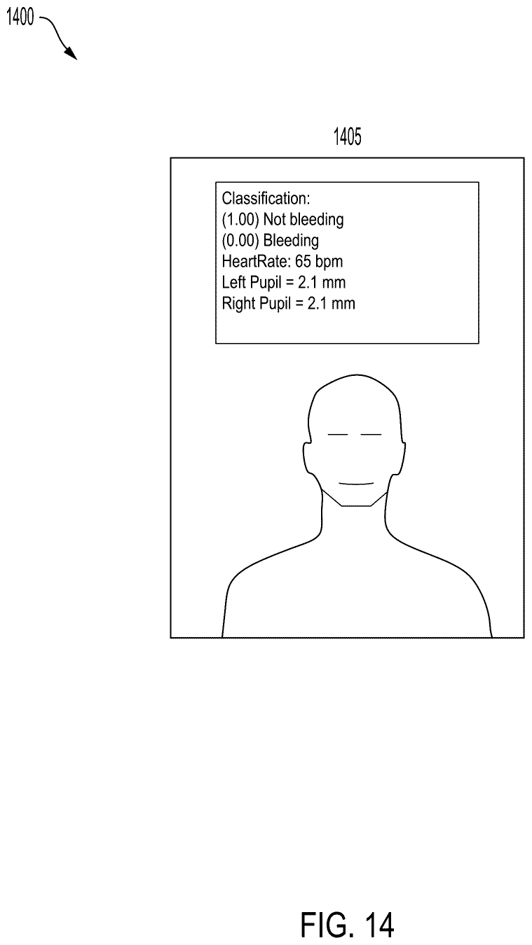

FIG. 14 depicts an example 1400 of a graphical user interface 1405 showing an image of a person along with data or information determined by the ML models 230 or 235. For example, user's image or video can be shown with the information about the classification determinations by the ML models, including pupil size, heart rate, determination on the bleeding or any other information. For example, the video or image in the graphical user interface 1405 can show how multiple AI or ML models can be integrated to extract video-based features such as pupil size and/or other camera-sensing changes such as photoplethysmography-based (PPG) signals such as heart rate, color, vascular, or micro-expression changes.

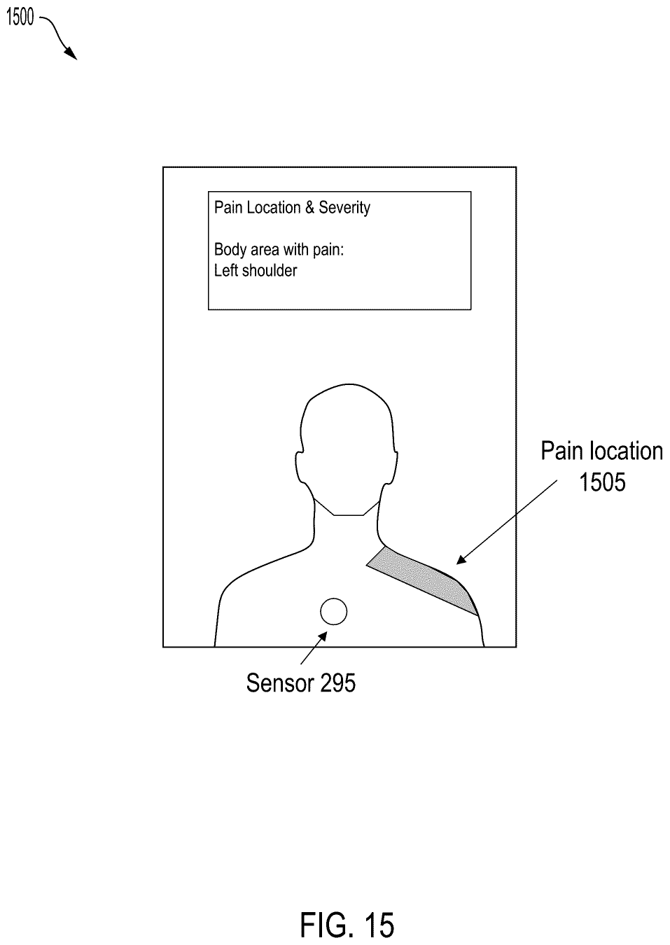

FIG. 15 depicts an example 1500 of a graphical user interface 1405 showing an image of a person along with data on pain location 1505 corresponding to the pain felt or experienced by the person, as determined by the ML models 230 or 235. For example, example 1500 can show location of a person's location of pain, as determined based on a sensor 295, such as a PPG or other sensor body wearables data that can be used to measure and provide vital signs to prospectively integrate into the AI or ML pain-related algorithms.

Some of the description herein emphasizes the structural independence of the aspects of the system components or groupings of operations and responsibilities of these system components. Other groupings that execute similar overall operations are within the scope of the present application. Modules can be implemented in hardware or as computer instructions on a non-transient computer readable storage medium, and modules can be distributed across various hardware or computer based components.

The systems described above can provide multiple ones of any or each of those components and these components can be provided on either a standalone system or on multiple instantiation in a distributed system. In addition, the systems and methods described above can be provided as one or more computer-readable programs or executable instructions embodied on or in one or more articles of manufacture. The article of manufacture can be cloud storage, a hard disk, a CD-ROM, a flash memory card, a PROM, a RAM, a ROM, or a magnetic tape. In general, the computer-readable programs can be implemented in any programming language, such as LISP, PERL, C, C++, C #, PROLOG, or in any byte code language such as JAVA. The software programs or executable instructions can be stored on or in one or more articles of manufacture as object code.

Example and non-limiting module implementation elements include sensors providing any value determined herein, sensors providing any value that is a precursor to a value determined herein, datalink or network hardware including communication chips, oscillating crystals, communication links, cables, twisted pair wiring, coaxial wiring, shielded wiring, transmitters, receivers, or transceivers, logic circuits, hard-wired logic circuits, reconfigurable logic circuits in a particular non-transient state configured according to the module specification, any actuator including at least an electrical, hydraulic, or pneumatic actuator, a solenoid, an op-amp, analog control elements (springs, filters, integrators, adders, dividers, gain elements), or digital control elements.

The subject matter and the operations described in this specification can be implemented in digital electronic circuitry, or in computer software, firmware, or hardware, including the structures disclosed in this specification and their structural equivalents, or in combinations of one or more of them. The subject matter described in this specification can be implemented as one or more computer programs, e.g., one or more circuits of computer program instructions, encoded on one or more computer storage media for execution by, or to control the operation of, data processing apparatuses. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. While a computer storage medium is not a propagated signal, a computer storage medium can be a source or destination of computer program instructions encoded in an artificially generated propagated signal. The computer storage medium can also be, or be included in, one or more separate components or media (e.g., multiple CDs, disks, or other storage devices include cloud storage). The operations described in this specification can be implemented as operations performed by a data processing apparatus on data stored on one or more computer-readable storage devices or received from other sources.

The terms “computing device”, “component” or “data processing apparatus” or the like encompass various apparatuses, devices, and machines for processing data, including by way of example a programmable processor, a computer, a system on a chip, or multiple ones, or combinations of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, app, script, or code) can be written in any form of programming language, including compiled or interpreted languages, declarative or procedural languages, and can be deployed in any form, including as a stand-alone program or as a module, component, subroutine, object, or other unit suitable for use in a computing environment. A computer program can correspond to a file in a file system. A computer program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatuses can also be implemented as, special purpose logic circuitry, e.g., an FPGA (field programmable gate array) or an ASIC (application specific integrated circuit). Devices suitable for storing computer program instructions and data can include non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

The subject matter described herein can be implemented in a computing system that includes a back end component, e.g., as a data server, or that includes a middleware component, e.g., an application server, or that includes a front end component, e.g., a client computer having a graphical user interface or a web browser through which a user can interact with an implementation of the subject matter described in this specification, or a combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

While operations are depicted in the drawings in a particular order, such operations are not required to be performed in the particular order shown or in sequential order, and all illustrated operations are not required to be performed. Actions described herein can be performed in a different order.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements and features discussed in connection with one implementation are not intended to be excluded from a similar role in other implementations or implementations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

Any implementation disclosed herein may be combined with any other implementation or embodiment, and references to “an implementation,” “some implementations,” “one implementation” or the like are not necessarily mutually exclusive and are intended to indicate that a particular feature, structure, or characteristic described in connection with the implementation may be included in at least one implementation or embodiment. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

Modifications of described elements and acts such as variations in sizes, dimensions, structures, shapes and proportions of the various elements, values of parameters, mounting arrangements, use of materials, colors, orientations can occur without materially departing from the teachings and advantages of the subject matter disclosed herein. For example, elements shown as integrally formed can be constructed of multiple parts or elements, the position of elements can be reversed or otherwise varied, and the nature or number of discrete elements or positions can be altered or varied. Other substitutions, modifications, changes and omissions can also be made in the design, operating conditions and arrangement of the disclosed elements and operations without departing from the scope of the present disclosure.

For example, descriptions of positive and negative electrical characteristics may be reversed. Further relative parallel, perpendicular, vertical or other positioning or orientation descriptions include variations within +/−10% or +/−10 degrees of pure vertical, parallel or perpendicular positioning. References to “approximately,” “substantially” or other terms of degree include variations of +/−10% from the given measurement, unit, or range unless explicitly indicated otherwise. Coupled elements can be electrically, mechanically, or physically coupled with one another directly or with intervening elements. Scope of the systems and methods described herein is thus indicated by the appended claims, rather than the foregoing description, and changes that come within the meaning and range of equivalency of the claims are embraced therein.

What is claimed is:

1. A system, comprising:
one or more processors coupled with memory to:
receive, from an application, a media of a person and data on health of the person;
identify, using one or more models, a presence of pain and a level of pain being expressed in the media of the person responsive to providing the media of the person and the data on health of the person as one or more inputs to the one or more models, the one or more models trained using a plurality of media and a plurality of data on health of persons expressing a plurality of levels of pain; and
generate, for the application, a notification identifying the presence of pain and the level of pain expressed by the person.

2. The system of claim 1, comprising the one or more processors to:
train the one or more models to identify the presence of pain using at least the plurality of media, wherein the plurality of media comprises at least one of a plurality of videos or a plurality of images of one or more body parts of a plurality of body parts of the plurality of persons expressing pain or not expressing pain.

3. The system of claim 1, comprising the one or more processors to:
train the one or more models to identify the level of pain based at least on the plurality of data, wherein the plurality of data comprises vital sign data of the plurality of persons experiencing the plurality of levels of pain.

4. The system of claim 1, comprising the one or more processors to:
determine the level of pain of a plurality of levels of pain of the person based at least on the data on health including at least one of a heart rate, temperature, oxygen level, respiratory rate, a systolic blood pressure and age of the person input into the one or more models.

5. The system of claim 1, comprising the one or more processors to:
determine, using the data on health input into a model of the one or more models comprising a neural network, at least one of the presence of pain or the level of pain, wherein the data on health comprises at least one of a medical history of the person or a measurement of a sensor attached to a body of the person.

6. The system of claim 1, comprising the one or more processors to:
identify, from the received media, at least one of a video or an image of the received media depicting a portion of a face of the person; and
identify, using the one or more models, at least one of the presence of pain or the level of pain responsive to providing the at least one of the video or the image as an input of the one or more inputs to the one or more models.

7. The system of claim 1, comprising the one or more processors to:
receive the media comprising a video capturing a movement of a plurality of parts of a body of the person; and
identify, using the one or more models, at least one of the presence of pain or the level of pain responsive to the movement.

8. The system of claim 1, comprising the one or more processors to:
receive the data on health comprising at least one of a prospective measurement or a retrospective measurement of a sensor of a wearable device of the person;
identify at least one of the presence of pain or the level of pain responsive to providing the at least one of the prospective measurement or the retrospective measurement as the one or more inputs to a model of the one or more models.

9. A method, comprising:
receiving, by a data processing system from an application, a media of a person and data on health of the person;
identifying, by one or more models of the data processing system, a presence of pain and a level of pain being expressed in the media of the person responsive to providing the media of the person and the data on health of the person as one or more inputs to the one or more models, the one or more models trained using a plurality of media and a plurality of data on health of persons expressing a plurality of levels of pain; and
generating, by the data processing system for the application, a notification identifying the presence of pain and the level of pain expressed by the person.

10. The method of claim 9, comprising:
training, by the data processing system, the one or more models to identify the presence of pain using at least the plurality of media, wherein the plurality of media comprises at least one of a plurality of videos or a plurality of images of one or more body parts of a plurality of body parts of the plurality of persons expressing pain or not expressing pain.

11. The method of claim 9, comprising:
training, by the data processing system, the one or more models to identify the level of pain based at least on the plurality of data on health, wherein the plurality of data on health comprises vital sign data of the plurality of persons experiencing the plurality of levels of pain.

12. The method of claim 9, comprising:
determining, using the one or more models, the level of pain of the person based at least on the data on health including at least one of a heart rate, temperature, oxygen level, respiratory rate, a systolic blood pressure and age of the person input into the one or more models.

13. The method of claim 9, comprising:
determining, using the data on health input into a model of the one or more models comprising a neural network, at least one of the presence of pain or the level of pain, wherein the data on health comprises at least one of a medical history of the person or a measurement of a sensor attached to a body of the person.

14. The method of claim 9, comprising:
identifying, by the data processing system from the received media, at least one of a video or an image of the received media depicting a portion of a face of the person; and
identifying, using the one or more models, at least one of the presence of pain or the level of pain responsive to providing the at least one of the video or the image as an input of the one or more inputs to the one or more models.

15. The method of claim 9, comprising:
receiving by the data processing system, the media comprising a video capturing a movement of a plurality of parts of a body of the person; and
identifying, using the one or more models, at least one of the presence of pain or the level of pain responsive to the movement.

16. The method of claim 9, comprising:
receiving, by the data processing system, the data on health comprising at least one of a prospective measurement or a retrospective measurement of a sensor of a wearable device of the person;
identifying, by the data processing system at least one of the presence of pain or the level of pain responsive to providing the at least one of the prospective measurement or the retrospective measurement as the one or more inputs to a model of the one or more models.

17. A non-transitory computer-readable media having processor readable instructions, such that, when executed, cause at least one processor to:
receive, from an application, a media of a person and data on health of the person;
identify, using one or more models, a presence of pain and a level of pain being expressed in the media of the person responsive to providing the media of the person and the data on health of the person as one or more inputs to the one or more models, the one or more models trained using a plurality of media and a plurality of data on health of persons expressing a plurality of levels of pain; and
generate, for the application, a notification identifying the presence of pain and the level of pain expressed by the person.

18. The non-transitory computer-readable media of claim 17, wherein the instructions, when executed, cause the at least one processor to:
train the one or more models to identify the presence of pain using at least the plurality of media, wherein the plurality of media comprises at least one of a plurality of videos or a plurality of images of one or more body parts of a plurality of body parts of the plurality of persons expressing pain and not expressing pain; and
train the one or more models to identify the level of pain based at least on the plurality of data, wherein the plurality of data comprises vital sign data of the plurality of persons experiencing the plurality of levels of pain.

19. The non-transitory computer-readable media of claim 17, wherein the instructions, when executed, cause the at least one processor to:
determine the level of pain of a plurality of levels of pain of the person based at least on the data on health including at least one of a heart rate, temperature, oxygen level, respiratory rate, a systolic blood pressure and age of the person input into the one or more models.

20. The non-transitory computer-readable media of claim 17, wherein the instructions, when executed, cause the at least one processor to:
determine, using the data on health input into a model of the one or more models comprising a neural network, at least one of the presence of pain or the level of pain, wherein the data on health comprises at least one of a medical history of the person or a measurement of a sensor attached to a body of the person.
